(19) United States
(12) Patent Application Publication
(10) Pub. No.: US 2017/0296032 A1
(43) Pub. Date: Oct. 19, 2017

(54) BRANCHING STRUCTURE DETERMINATION APPARATUS, METHOD, AND PROGRAM

(71) Applicant: FUJIFILM Corporation, Tokyo (JP)

(72) Inventor: Yuanzhong LI, Tokyo (JP)

(73) Assignee: FUJIFILM Corporation, Tokyo (JP)

(21) Appl. No.: 15/639,067

(22) Filed: Jun. 30, 2017

Related U.S. Application Data
(63) Continuation of application No. PCT/JP2016/001161, filed on Mar. 3, 2016.

(30) Foreign Application Priority Data
Mar. 6, 2015 (JP) 2015-044177

(51) Int. Cl. A61B 1/00; A61B 5/00; A61B 1/267; A61B 1/04; G06T 7/00

(52) U.S. Cl. CPC A61B 1/00009; A61B 5/7425; A61B 1/0005; A61B 1/2676; A61B 1/04; A61B 34/20

(57) ABSTRACT

An image acquisition unit acquires a real endoscope image. A first evaluation value acquisition unit acquires a first evaluation value indicating the hole likeness of the insertion destination of an endoscope, and a second evaluation value acquisition unit acquires a second evaluation value indicating the boundary likeness of a plurality of bronchi at the bronchial branch. A determination unit determines whether or not a branching structure is included in the real endoscope image using both the first and second evaluation values. When it is determined that a branching structure is included, a virtual endoscope image generation unit generates a virtual endoscope image, and a display control unit displays the real endoscope image and the virtual endoscope image on a display.

### CROSS REFERENCE TO RELATED APPLICATIONS

The present application is a Continuation of PCT International Application No. PCT/JP2016/001161 filed on Mar. 3, 2016, which claims priority under 35 U.S.C. §119(a) to Japanese Patent Application No. 2015-044177 filed on Mar. 6, 2015. Each of the above applications is hereby expressly incorporated by reference, in its entirety, into the present application.

### BACKGROUND

### Technical Field

The present invention relates to a branching structure determination apparatus, method, and program for determining a branching structure at a branching position of a tubular structure having a branching structure, such as a bronchus, using an endoscope image that is acquired by inserting an endoscope into the tubular structure and performing imaging.

### Description of the Related Art

In recent years, a technique of observing or treating a tubular structure, such as the large intestine or bronchus of a patient, using an endoscope has been drawing attention. However, in the endoscope image, an image in which the color or texture of the inside of the tubular structure is clearly expressed by an imaging element, such as a charge coupled device (CCD), can be obtained, while the inside of the tubular structure is expressed as a two-dimensional image. For this reason, it is difficult to ascertain which position in the tubular structure the endoscope image represents. In particular, since a bronchial endoscope has a small diameter and accordingly has a narrow field of view, it is difficult to make the distal end of the endoscope reach a target position.

Therefore, a method has been proposed in which a virtual endoscope image similar to an image that has been actually captured by an endoscope is generated using a three-dimensional image acquired by tomographic imaging using a modality, such as a computed tomography (CT) apparatus or a magnetic resonance imaging (MRI) apparatus. The virtual endoscope image is used as a navigation image for guiding the endoscope to a target position in the tubular structure. However, even if the navigation image is used, in the case of a structure having a pipe line branching in multiple stages, such as a bronchus, a skilled technique is required to make the distal end of the endoscope reach a target position in a short time.

For this reason, a method has been proposed in which an image of a tubular structure is extracted from a three-dimensional image, matching between the image of the tubular structure and a real endoscope image, which is a real endoscope image acquired by performing imaging with an endoscope, is performed, and a virtual endoscope image at the current position of the endoscope is generated from the three-dimensional image of the tubular structure and is displayed (refer to JP2013-150650A).

In the method disclosed in JP2013-150650A, however, it is necessary to perform matching between the real endoscope image and the entire three-dimensional image of the tubular structure. Accordingly, a long processing time is required. On the other hand, in order to make the endoscope reach the target position, it is important to advance the endoscope to a portion of the tubular structure connected to the target position at a branching position in the tubular structure. For this reason, a method of recognizing a branching position in a real endoscope image has been proposed (refer to JP2012-505695A and JP2012-531932A). JP2012-505695A discloses a method in which an image feature representing a branching position is detected from a real endoscope image using a known shape descriptor and matching with a virtual endoscope image is performed based on the detected image feature to acquire a virtual endoscope image corresponding to the branching position. JP2012-531932A has proposed a method of detecting a branching position from a real endoscope image by image processing and generating an image of a branching position of a virtual endoscope using the detected branching position.

If a branching position is detected in the real endoscope image using the methods disclosed in JP2012-505695A and JP2012-531932A, it is sufficient to perform matching only in the vicinity of the branching position in the image of the tubular structure. Accordingly, it is possible to quickly generate a virtual endoscope image corresponding to a real endoscope image acquired by the endoscope that has reached the branching position.

### SUMMARY

In the method disclosed in JP2012-505695A, the branching position is detected using the shape of the branching structure. However, there is no description in JP2012-505695A as to what shape feature of the branching structure is used at the branching position. In addition, JP2012-531932A discloses a configuration for detecting a branching position by image processing. However, there is no description in JP2012-531932A as to specifically what kind of image processing is used to detect a branching position. As described above, in the methods disclosed in JP2012-505695A and JP2012-531932A, how to detect a branching structure at the branching position is not clear. Accordingly, there is a possibility that a branching structure cannot be detected with high accuracy.

The present invention has been made in view of the above circumstances, and it is an object of the present invention to accurately determine whether or not a branching structure of a tubular structure is included in a real endoscope image in a branching structure determination apparatus, method, and program.

A branching structure determination apparatus according to the present invention comprises: real endoscope image acquisition unit for acquiring a real endoscope image that is generated by performing imaging using an endoscope inserted into a tubular structure having a branching structure in a subject and that shows an inner wall of the tubular structure; first evaluation value acquisition unit for acquiring a first evaluation value, which indicates hole likeness of an insertion destination of the endoscope in the tubular structure, from the real endoscope image; second evaluation value acquisition unit for acquiring a second evaluation value, which indicates boundary likeness of a plurality of tubular structures at a branch of the tubular structure, from the real endoscope image; and determination unit for determining whether or not the branching structure is included in the real endoscope image using both the first and second evaluation values.

In the real endoscope image acquired by the endoscope inserted into the tubular structure, the inner part of the tubular structure is dark since the light of the endoscope does not reach there. The subsequent destination to which the endoscope is to be inserted looks like a deep hole. The “first evaluation value indicating the hole likeness of the insertion destination of the endoscope” unit an evaluation value indicating how deep the hole appears in the real endoscope image. On the other hand, at the branching position where a plurality of tubular structures are connected to each other, in a case where the connected tubular structures are cut along the center line, the cross section of a boundary portion of the connected tubular structures is a ridge shape. In the real endoscope image, the boundary portion has a linear structure interposed between deep holes. The “second evaluation value indicating the boundary likeness of a plurality of tubular structures” unit an evaluation value indicating how much it looks like a boundary in the real endoscope image.

In the branching structure determination apparatus according to the present invention, the determination unit may acquire a candidate for a hole into which the endoscope is to be inserted based on the first evaluation value, acquire a candidate for a boundary of a plurality of tubular structures at a branch of the tubular structure based on the second evaluation value, and determine whether or not the branching structure is included in the real endoscope image based on the hole candidate and the boundary candidate.

In the branching structure determination apparatus according to the present invention, the determination unit may calculate a weighted average of the first evaluation value, which is the hole candidate, and the second evaluation value, which is the boundary candidate, and determine that the branching structure is included in the real endoscope image in a case where the weighted average is equal to or greater than a predetermined threshold value.

In the branching structure determination apparatus according to the present invention, the first evaluation value acquisition unit may calculate the first evaluation value using a learning result acquired by machine-learning the hole likeness of the insertion destination of the endoscope.

In the branching structure determination apparatus according to the present invention, the second evaluation value acquisition unit may calculate the second evaluation value using a learning result acquired by machine-learning the boundary likeness of the plurality of tubular structures.

The branching structure determination apparatus according to the present invention may further comprise real endoscope image display unit for displaying the real endoscope image.

The branching structure determination apparatus according to the present invention may further comprise emphasis unit for emphasizing the branching structure in the displayed real endoscope image in a case where it is determined that the branching structure is included.

In this case, the emphasis unit may emphasize the branching structure by giving a marker to at least one of a hole of the insertion destination of the endoscope in the tubular structure or a boundary of the plurality of tubular structures.

The “marker” is for emphasizing at least one of the hole of the insertion destination of the endoscope or the boundary of the plurality of tubular structures. For example, a line surrounding a hole and a line indicating a boundary can be used as a marker. Instead of the lines, symbols and letters indicating holes and boundaries can also be used as markers. Color may be given to the marker, or the display of the marker may be blinked.

The branching structure determination apparatus according to the present invention may further comprise warning unit for giving a warning in a case where it is determined that the branching structure is included.

The branching structure determination apparatus according to the present invention may further comprise: three-dimensional image acquisition unit for acquiring a three-dimensional image including the tubular structure of the subject; and virtual endoscope image generation unit for generating a virtual endoscope image, which shows an inner wall of the tubular structure in a case where the branching structure is viewed at a position in the three-dimensional image corresponding to a position of a distal end of the endoscope where a real endoscope image determined to include the branching structure is acquired, from the three-dimensional image in a case where it is determined that the branching structure is included.

In this case, the branching structure determination apparatus according to the present invention may further comprise virtual endoscope image display unit for displaying the virtual endoscope image.

In addition, in this case, the virtual endoscope image generation unit may extract the tubular structure from the three-dimensional image, and specify a position of a distal end of the endoscope in the extracted tubular structure. The branching structure determination apparatus according to the present invention may further comprise tubular structure display unit for displaying an image of the extracted tubular structure.

In the branching structure determination apparatus according to the present invention, the tubular structure display unit may further display the specified position of the distal end of the endoscope in the displayed tubular structure.

A branching structure determination method according to the present invention comprises: acquiring a real endoscope image that is generated by performing imaging using an endoscope inserted into a tubular structure having a branching structure in a subject and that shows an inner wall of the tubular structure; acquiring a first evaluation value, which indicates hole likeness of an insertion destination of the endoscope in the tubular structure, from the real endoscope image; acquiring a second evaluation value, which indicates boundary likeness of a plurality of tubular structures at a branch of the tubular structure, from the real endoscope image; and determining whether or not the branching structure is included in the real endoscope image using both the first and second evaluation values.

In addition, a program causing a computer to execute the branching structure determination method according to the present invention may be provided.

According to the present invention, the first evaluation value indicating the hole likeness of the insertion destination of the endoscope in the tubular structure is acquired from the real endoscope image, and the second evaluation value indicating the boundary likeness of a plurality of bronchi at the branch of the tubular structure is acquired from the real endoscope image. Then, it is determined whether or not a branching structure is included in the real endoscope image using both the first and second evaluation values. Here, the first and second evaluation values remarkably indicate the feature of the branching position. Therefore, according to the present invention, it is possible to accurately determine whether or not a branching structure is included in the real endoscope image using the first and second evaluation values.

### BRIEF DESCRIPTION OF THE DRAWINGS

FIG. 1 is a hardware configuration diagram showing an outline of a diagnosis assistance system to which a branching structure determination apparatus according to an embodiment of the present invention is applied.

FIG. 2 is a diagram showing the schematic configuration of the branching structure determination apparatus realized by installing a branching structure determination program in a computer.

FIG. 3 is a diagram showing a real endoscope image at the branching position of a bronchus.

FIG. 4 is a diagram showing a real endoscope image at the branching position of a bronchus.

FIG. 5 is a cross-sectional view at the branching position of a bronchus.

FIG. 6 is a diagram illustrating the matching.

FIG. 7 is a diagram showing a real endoscope image and a virtual endoscope image that are displayed on a display.

FIG. 8 is a diagram showing a bronchus image, a real endoscope image, and a virtual endoscope image that are displayed on the display.

FIG. 9 is a diagram illustrating the emphasis of a branching structure and warning in the real endoscope image.

FIG. 10 is a diagram illustrating the emphasis of a branching structure in the real endoscope image.

FIG. 11 is a flowchart showing a process performed in the present embodiment.

### DETAILED DESCRIPTION

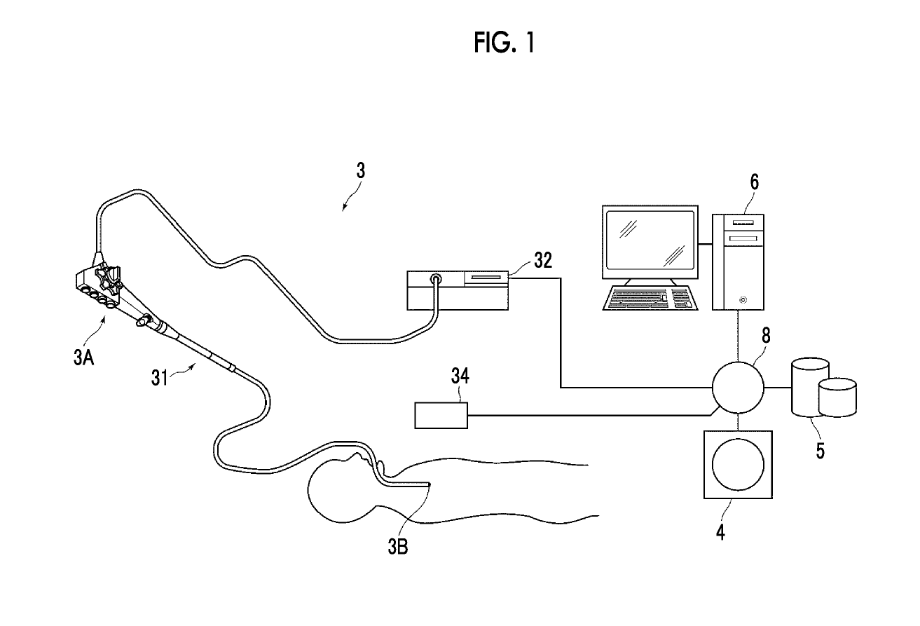

Hereinafter, an embodiment of the present invention will be described with reference to the diagrams. FIG. 1 is a hardware configuration diagram showing an outline of a diagnosis assistance system to which a branching structure determination apparatus according to the embodiment of the present invention is applied. As shown in FIG. 1, in this system, an endoscope apparatus 3, a three-dimensional image capturing apparatus 4, an image storage server 5, and a branching structure determination apparatus 6 are connected to each other in a communicable state through a network 8.

The endoscope apparatus 3 includes an endoscope scope 31 for imaging the inside of a tubular structure of a subject, a processor device 32 for generating an image of the inside of the tubular structure based on a signal obtained by imaging, a position detection device 34 for detecting the position and direction of the distal end of the endoscope scope 31.

The endoscope scope 31 is obtained by continuously attaching an insertion part, which is inserted into the tubular structure of the subject, to an operation unit 3A, and is connected to the processor device 32 through a universal cord detachably connected to the processor device 32. The operation unit 3A includes various buttons for giving an instruction of an operation to make a distal end 3B of the insertion part curve in a vertical direction and a horizontal direction within a predetermined angular range or for collecting samples of tissues by operating an insertion needle attached to the distal end of the endoscope scope 31. In the present embodiment, the endoscope scope 31 is a flexible mirror for bronchi, and is inserted into the bronchus of the subject. Then, light guided through an optical fiber from a light source device (not shown) provided in the processor device 32 is emitted from the distal end 3B of the insertion part of the endoscope scope 31, and an image of the inside of the bronchus of the subject is acquired by the imaging optical system of the endoscope scope 31. In order to facilitate the description, the distal end 3B of the insertion part of the endoscope scope 31 will be referred to as the endoscope distal end 3B in the following description.

The processor device 32 generates an endoscope image T0 by converting an imaging signal captured by the endoscope scope 31 into a digital image signal and correcting image quality by digital signal processing, such as white balance adjustment and shading correction. The generated image is a motion picture expressed at a predetermined frame rate, such as 30 fps, for example. The endoscope image T0 is transmitted to the image storage server 5 or the branching structure determination apparatus 6. In the following description, the endoscope image T0 captured by the endoscope apparatus 3 is referred to as a real endoscope image T0 in order to distinguish it from a virtual endoscope image to be described later.

The position detection device 34 detects the position and direction of the endoscope distal end 3B in the body of the subject. Specifically, the relative position and direction of the endoscope distal end 3B in the body of the subject are detected by detecting the characteristic shape of the endoscope distal end 3B with an echo device having a detection region of the three-dimensional coordinate system in which the position of a specific part of the subject is a reference point, and information of the detected position and direction of the endoscope distal end 3B is output to the branching structure determination apparatus 6 as position information (for example, refer to JP2006-61274A). The detected position and direction of the endoscope distal end 3B correspond to a viewpoint and an eye direction of an endoscope image obtained by imaging, respectively. In the following description, the position and direction information is simply referred to as position information. The position information is output to the branching structure determination apparatus 6 at a rate similar to that of the real endoscope image T0.

The three-dimensional image capturing apparatus 4 is an apparatus that generates a three-dimensional image V0 showing a part, which is an examination target part of a subject, by imaging the part. Specifically, the three-dimensional image capturing apparatus 4 is a CT apparatus, an MRI apparatus, a positron emission tomography (PET) apparatus, an ultrasound diagnostic apparatus, or the like. The three-dimensional image V0 generated by the three-dimensional image capturing apparatus 4 is transmitted to the image storage server 5 and is stored therein. In the present embodiment, the three-dimensional image capturing apparatus 4 generates the three-dimensional image V0 by imaging the chest including the bronchus.

The image storage server 5 is a computer that stores and manages various kinds of data, and includes a large-capacity external storage device and software for database management. The image storage server 5 transmits and receives image data and the like by performing communication with other apparatuses through the network 8. Specifically, the image storage server 5 acquires image data, such as the real endoscope image T0 acquired by the endoscope apparatus 3 and the three-dimensional image V0 generated by the three-dimensional image capturing apparatus 4, through the network, and stores the image data in a recording medium, such as a large-capacity external storage device and manages the image data. The real endoscope image T0 is motion picture data captured according to the movement of the endoscope distal end 3B. Therefore, it is preferable that the real endoscope image T0 is transmitted to the branching structure determination apparatus 6 without passing through the image storage server 5. The storage format of image data or the communication between apparatuses through the network 8 is based on protocols, such as a digital imaging and communication in medicine (DICOM).

The branching structure determination apparatus 6 is realized by installing a branching structure determination program of the present invention in one computer. The computer may be a workstation or a personal computer that is directly operated by a doctor who performs diagnosis, or may be a server computer connected to these through a network. The branching structure determination program is distributed by being recorded on a recording medium, such as a digital versatile disc (DVD) or a compact disk read only memory (CD-ROM), and is installed into the computer from the recording medium. Alternatively, the branching structure determination program is stored in a storage device of a server computer connected to the network or in a network storage so as to be accessible from the outside, and is downloaded and installed into a computer used by a doctor, who is a user of the branching structure determination apparatus 6, when necessary.

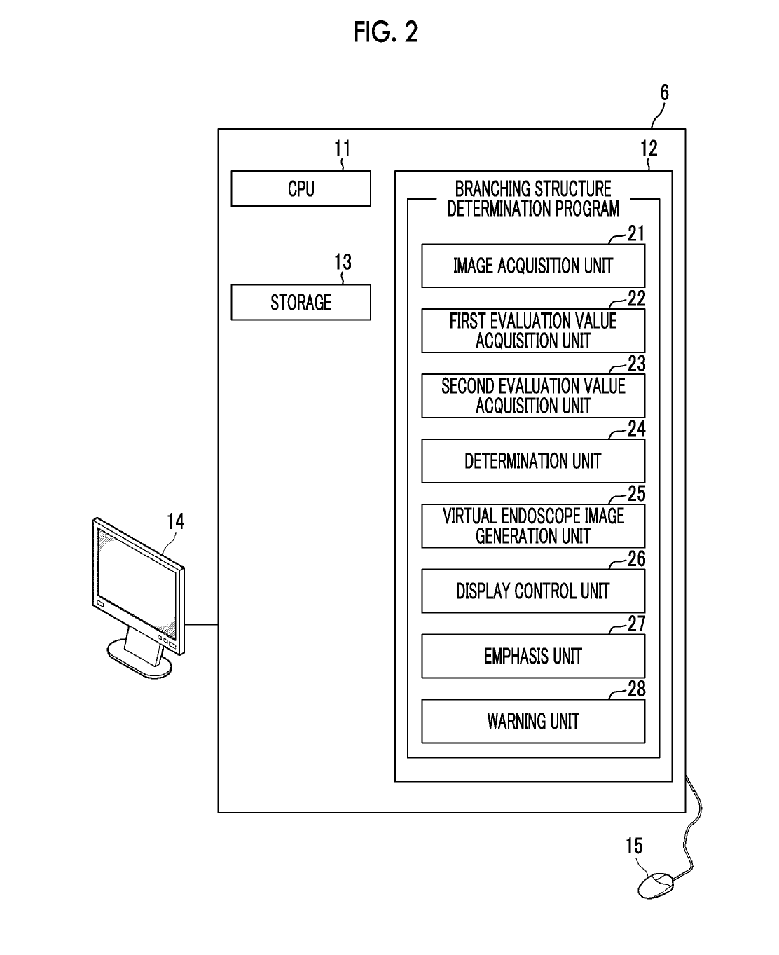

FIG. 2 is a diagram showing the schematic configuration of a branching structure determination apparatus realized by installing a branching structure determination program in a computer. As shown in FIG. 2, the branching structure determination apparatus 6 includes a central processing unit (CPU) 11, a memory 12, and a storage 13 as the configuration of a standard workstation. A display 14 and an input unit 15, such as a mouse, are connected to the branching structure determination apparatus 6.

The real endoscope image T0 and the three-dimensional image V0, which are acquired from the endoscope apparatus 3, the three-dimensional image capturing apparatus 4, the image storage server 5, and the like through the network 8, and the image generated by the processing in the branching structure determination apparatus 6, and the like are stored in the storage 13.

A branching structure determination program is stored in the memory 12. The branching structure determination program specifies, as processing to be executed by the CPU 11: image acquisition processing for acquiring image data, such as the real endoscope image T0 generated by the processor device 32 and the three-dimensional image V0 generated by the three-dimensional image capturing apparatus 4; first evaluation value acquisition processing for acquiring a first evaluation value, which indicates the hole likeness of an insertion destination of the endoscope in the bronchus that is a tubular structure, from the real endoscope image T0; second evaluation value acquisition processing for acquiring a second evaluation value, which indicates the boundary likeness of a plurality of bronchi at the bronchial branch, from the real endoscope image T0; determination processing for determining whether or not a branching structure is included in the real endoscope image T0 using both the first and second evaluation values; virtual endoscope image generation processing for generating a virtual endoscope image from the three-dimensional image V0 in a case where it is determined that a branching structure is included; display control processing for displaying the real endoscope image T0 and the virtual endoscope image; emphasis processing for emphasizing the branching structure in the real endoscope image T0 displayed on the display 14 in a case where it is determined that a branching structure is included; and warning processing for giving a warning in a case where it is determined that a branching structure is included.

The CPU 11 executes these processes according to the program, so that the computer functions as an image acquisition unit 21, a first evaluation value acquisition unit 22, a second evaluation value acquisition unit 23, a determination unit 24, a virtual endoscope image generation unit 25, a display control unit 26, an emphasis unit 27, and a warning unit 28. The branching structure determination apparatus 6 may include a plurality of processors that perform image acquisition processing, first evaluation value acquisition processing, second evaluation value acquisition processing, determination processing, virtual endoscope image generation processing, display control processing, emphasis processing, and warning processing. Here, the image acquisition unit 21 corresponds to real endoscope image acquisition unit and three-dimensional image acquisition unit, and the display 14 corresponds to real endoscope image display unit, virtual endoscope image display unit, and tubular structure display unit.

The image acquisition unit 21 acquires the real endoscope image T0 and the three-dimensional image V0 by imaging the inside of the bronchus at a predetermined viewpoint position with the endoscope apparatus 3. In a case where the real endoscope image T0 and the three-dimensional image V0 are already stored in the storage 13, the image acquisition unit 21 may acquire the real endoscope image T0 and the three-dimensional image V0 from the storage 13. The real endoscope image T0 is an image showing the inner surface of the bronchus, that is, the inner wall of the bronchus. The real endoscope image T0 is output to the display control unit 26, and is displayed on the display 14.

The first evaluation value acquisition unit 22 acquires a first evaluation value E1, which indicates the hole likeness of the insertion destination of the endoscope, from the real endoscope image T0. Specifically, a feature quantity indicating the hole likeness in the real endoscope image T0 is acquired as the first evaluation value E1. Therefore, the first evaluation value acquisition unit 22 includes a discriminator that is a learning result generated by machine-learning an image of a hole at the branching position of the bronchus as a teacher image.

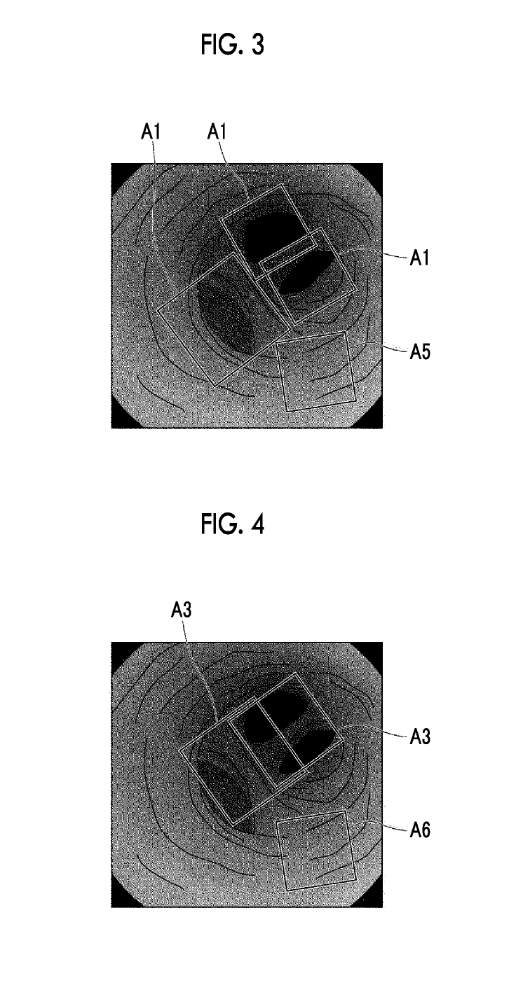

FIG. 3 is a diagram showing a real endoscope image at the branching position of a bronchus. As shown in FIG. 3, at the branching position, the inner wall of the bronchus can be visually recognized in a range where light emitted from the distal end 3B of the endoscope reaches. In the subsequent destination to which the endoscope is to be inserted, a dark and deep hole is viewed since light does not reach there. In learning of a discriminator for acquiring the first evaluation value E1, an image of a hole region cut out from a sample image is used as a teacher image A1 as shown in FIG. 3. The teacher image A1 is a square image standardized such that the center of the hole is at the center of gravity, the long axis of the hole is horizontal, and the length of the long axis of the hole is a predetermined length. For learning, a teacher image (referred to as A2) other than the hole is also prepared. Then, by performing learning using a machine learning algorithm, such as boosting, with the teacher image A1 of the hole as a positive teacher image and the teacher image A2 other than the hole as a negative teacher image, a discriminator for the first evaluation value E1 is acquired. The discriminator outputs a score for the input image. The larger the score, the higher the possibility that the input image includes the branch hole. As a machine learning algorithm, for example, a method described in “Robust Real-Time Face Detection, International Journal of Computer Vision 57(2), 137-154, 2004.” may be used.

The second evaluation value acquisition unit 23 acquires a second evaluation value E2, which indicates the boundary likeness of the bronchus at the branch, from the real endoscope image T0. Specifically, a feature quantity indicating the boundary likeness in the real endoscope image T0 is acquired as the second evaluation value E2. Therefore, the second evaluation value acquisition unit 23 includes a discriminator that is a learning result generated by machine-learning an image of a boundary at the branching position of the bronchus as a teacher image.

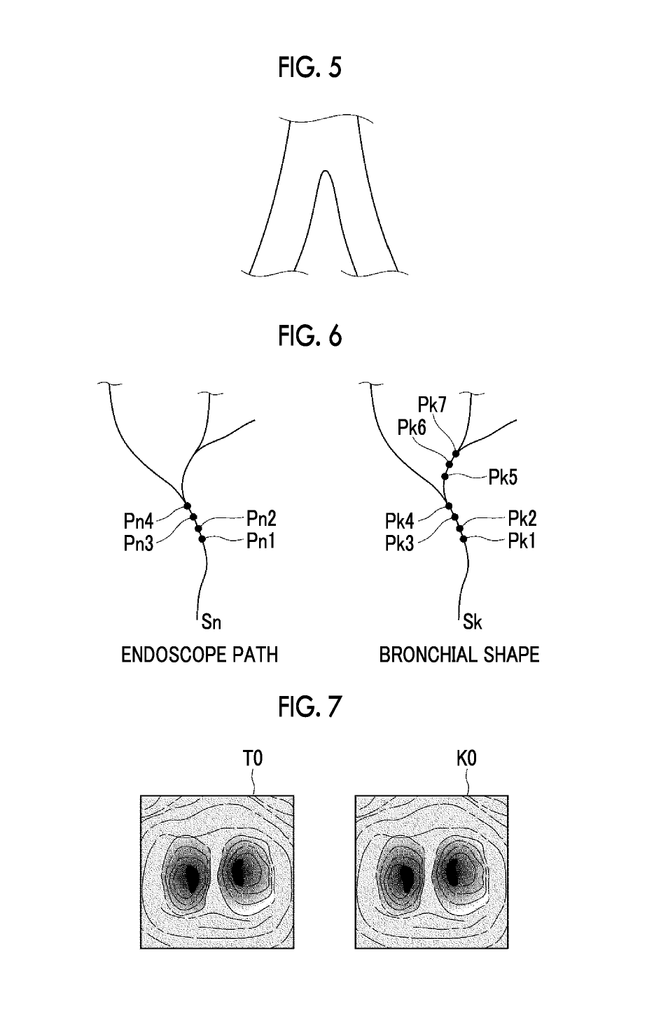

FIG. 4 is a diagram showing a real endoscope image at the branching position of a bronchus similar to that in FIG. 3. As shown in FIG. 4, at the branching position, the inner wall of the bronchus can be visually recognized in a range where light emitted from the distal end 3B of the endoscope reaches. In the subsequent destination to which the endoscope is to be inserted, a dark and deep hole is viewed since light does not reach there. Between deep holes, the boundary of the holes is viewed. Here, the boundary of the holes is a ridge shape as shown in the cross-sectional view of the branching position of FIG. 5. Therefore, the boundary of the holes has a linear structure interposed between the two holes. In learning of a discriminator for acquiring the second evaluation value E2, an image of a boundary region cut out from a sample image is used as a teacher image A3 as shown in FIG. 4. The teacher image A3 is a square image standardized such that the center of a linear portion of the boundary is at the center of gravity, the boundary portion is horizontal, and the length of the boundary portion is a predetermined length. For learning, a teacher image (referred to as A4) other than the boundary is also prepared. Then, by performing learning using a machine learning algorithm, such as boosting, with the teacher image A3 of the boundary as a positive teacher image and the teacher image A4 other than the boundary as a negative teacher image, a discriminator for the second evaluation value E2 is acquired. The discriminator outputs a score for the input image. The larger the score, the higher the possibility that the input image includes the boundary of the branch.

The first evaluation value acquisition unit 22 cuts out a square region from the real endoscope image T0, and inputs the cut region to the discriminator for the first evaluation value. The discriminator for the first evaluation value outputs a score of the hole likeness for the cut region. The first evaluation value acquisition unit 22 outputs a plurality of scores by cutting out regions of different positions, different sizes, and different rotation angles in the real endoscope image T0 and repeating the above-described determination using the cut-out regions. As regions having different sizes, for example, regions of ten sizes of 10 pixels×10 pixels to 100 pixels×100 pixels may be cut out at intervals of 10 pixels at the same position. As regions having different rotation angles, for example, regions of twelve rotation angles may be cut out at intervals of 30° at the same position and the same size.

Here, as shown in FIG. 3, in a case where the cut region matches the positive teacher image, the score increases. However, in a case where the cut region does not match the positive teacher image as shown in a region A5 in FIG. 3, the score decreases. The first evaluation value acquisition unit 22 acquires the score output from the discriminator as the first evaluation value E1.

On the other hand, similarly to the first evaluation value acquisition unit 22, the second evaluation value acquisition unit 23 cuts out a square region from the real endoscope image T0, and inputs the cut region to the discriminator for the second evaluation value. The discriminator for the second evaluation value outputs a score of the boundary likeness for the cut region. The second evaluation value acquisition unit 23 outputs a plurality of scores by cutting out regions of different positions, different sizes, and different rotation angles in the real endoscope image T0 and repeating the above-described determination using the cut-out regions.

Here, as shown in FIG. 4, in a case where the cut region matches the positive teacher image, the score increases. However, in a case where the cut region does not match the positive teacher image as shown in a region A6 in FIG. 4, the score decreases. The second evaluation value acquisition unit 23 acquires the score output from the discriminator as the second evaluation value E2.

The determination unit 24 determines whether or not a branching structure of the bronchus is included in the real endoscope image T0 using both the first evaluation value E1 and the second evaluation value E2. First, the determination unit 24 determines whether or not there are two or more holes in the real endoscope image T0 and there is a boundary between the holes. For this purpose, the determination unit 24 acquires, as the position of a hole candidate, a position where the first evaluation value E1 equal to or greater than a threshold value Th1 is acquired. In addition, the determination unit 24 acquires, as the position of a boundary candidate, a position where the second evaluation value E2 equal to or greater than a threshold value Th2 is acquired. Then, it is determined whether or not two or more hole candidates have been acquired and a boundary candidate has been acquired at a position therebetween. It may be determined whether or not a hole candidate has been acquired on each of both sides of the position where the boundary candidate is acquired. Then, in a case where the determination is positive, a weighted average of the first evaluation value E1 that is a hole candidate and the second evaluation value E2 that is a boundary candidate is calculated. In a case where the weighted average is equal to or greater than a predetermined threshold value Th3, it is determined that a branching structure of the bronchus is included in the real endoscope image T0. In this case, in a case where the second evaluation value E2 that is a boundary candidate is present between the two first evaluation values E1 that are hole candidates, the weight of the weighted average may be determined so that the value of the weighted average increases. Thus, in a case where it is determined that a branching structure is included, the position where the first evaluation value E1 that is a hole candidate is acquired can be detected as the position of the hole in the branch. In addition, the position where the second evaluation value E2 that is a boundary candidate is acquired can be detected as the position of the boundary in the branch.

The determination performed by the determination unit 24 is not limited to using the weighted average, and an average of the first evaluation value E1 and the second evaluation value E2 may be used or an addition value of the first evaluation value E1 and the second evaluation value E2 may be used. A multiplication value of the first evaluation value E1 and the second evaluation value E2 may be used. Only by determining whether or not two or more hole candidates have been acquired and a boundary candidate has been acquired at a position therebetween without calculating the weighted average, it may be determined whether or not a branching structure is included. In addition, only by determining whether or not a hole candidate has been acquired on each of both sides of the position where the boundary candidate is acquired, it may be determined whether or not a branching structure is included.

In a case where it is determined that a branching structure is included in the real endoscope image T0, the virtual endoscope image generation unit 25 generates a virtual endoscope image K0, which shows an inner wall of the bronchus as viewed from the viewpoint of the three-dimensional image V0 corresponding to the viewpoint of the real endoscope image T0, from the three-dimensional image V0. Hereinafter, the generation of the virtual endoscope image K0 will be described.

First, the virtual endoscope image generation unit 25 extracts a bronchus from the three-dimensional image V0. Specifically, the virtual endoscope image generation unit 25 extracts a graph structure of a bronchial region included in the input three-dimensional image V0, as a three-dimensional bronchus image, using the method disclosed in JP2010-220742A or the like, for example. Hereinafter, an example of the graph structure extraction method will be described.

In the three-dimensional image V0, pixels inside the bronchus are expressed as a region showing a low pixel value since the pixels correspond to an air region. However, the bronchial wall is expressed as a cylindrical or linear structure showing relatively high pixel values. Therefore, structural analysis of the shape based on the distribution of pixel values is performed for each pixel to extract the bronchus.

The bronchus branches in multiple stages, and the diameter of the bronchus decreases as the distance from the end decreases. The virtual endoscope image generation unit 25 generates a plurality of three-dimensional images with different resolutions by performing multi-resolution conversion of the three-dimensional image V0 so that bronchi having different sizes can be detected, and applies a detection algorithm for each three-dimensional image of each resolution, thereby detecting tubular structures having different sizes.

First, at each resolution, a Hessian matrix of each pixel of the three-dimensional image is calculated, and it is determined whether or not the pixel is a pixel in the tubular structure from the magnitude relationship of eigenvalues of the Hessian matrix. The Hessian matrix is a matrix having, as its elements, partial differential coefficients of the second order of density values in the respective axes (x, y, and z axes of the three-dimensional image), and is a 3×3 matrix as in the following equation.

$${∇}^{2} I = [ \begin{matrix}{I}_{xx} & {I}_{xy} & {I}_{xz} \\ {I}_{xx} & {I}_{xy} & {I}_{xz} \\ {I}_{xx} & {I}_{xy} & {I}_{xz}\end{matrix} ] {I}_{xx} = \frac{{δ}^{2} I}{δ {x}^{2}} , {I}_{xy} = \frac{{δ}^{2} I}{δ x δ {y}^{2}} , … \qquad [ Equation 1 ]$$

Assuming that the eigenvalues of the Hessian matrix at an arbitrary pixel are λ1, λ2, and λ3, in a case where two of the eigenvalues are large and one eigenvalue is close to 0, for example, when λ3, Δ2>>λ1 and λ1≅0 are satisfied, it is known that the pixel is a tubular structure. In addition, an eigenvector corresponding to the minimum eigenvalue (λ1≅0) of the Hessian matrix matches a main axis direction of the tubular structure.

The bronchus can be expressed in a graph structure, but the tubular structure extracted in this manner is not necessarily detected as one graph structure, in which all tubular structures are connected to each other, due to the influence of a tumor or the like. Therefore, after the detection of the tubular structure from the three-dimensional image V0 is ended, by performing evaluation regarding whether each extracted tubular structure is within a predetermined distance and an angle between the direction of the basic line connecting arbitrary points on the extracted two tubular structures to each other and the main axis direction of each tubular structure is within a predetermined angle, it is determined whether or not a plurality of tubular structures are connected to each other, thereby reconstructing the connection relationship of the extracted tubular structures. By this reconstruction, the extraction of the graph structure of the bronchus is completed.

Then, the virtual endoscope image generation unit 25 classifies the extracted graph structure into a start point, an end point, a branch point, and a side and connecting the start point, the end point, and the branch point to each other with the side, thereby being able to obtain a three-dimensional graph structure showing the bronchi as a bronchus image. The method of generating a graph structure is not limited to the method described above, and other methods may be adopted.

The virtual endoscope image generation unit 25 performs matching between the bronchus image and the real endoscope image T0. For the matching, for example, it is possible to use the method disclosed in JP2013-150650A. Here, the matching is processing for performing registration between the bronchus shown by the bronchus image and the actual position in the bronchus of the endoscope distal end 3B. Therefore, the virtual endoscope image generation unit 25 acquires the path information of the endoscope distal end 3B in the bronchus. Specifically, a line segment obtained by approximating the position of the endoscope distal end 3B detected by the position detection device 34 with a spline curve or the like is acquired as the path information. Then, as shown in FIG. 6, matching candidate points Pn1, Pn2, Pn3, . . . are set on the endoscope path at sufficiently short range intervals of about 5 mm to 1 cm, and matching candidate points Pk1, Pk2, Pk3, . . . are set on the bronchial shape at the same range intervals. In the present embodiment, the virtual endoscope image generation unit 25 generates the virtual endoscope image K0 in a case where it is determined that a branching structure is included in the real endoscope image T0. Therefore, the virtual endoscope image generation unit 25 sets matching candidate points in the bronchus only in the vicinity of the branching position in the bronchus image. For the endoscope path, matching candidate points are set only in a predetermined range before the current position.

Then, the virtual endoscope image generation unit 25 performs matching by associating the matching candidate points in the endoscope path and the matching candidate points of the bronchial shape in order from endoscope insertion positions Sn and Sk. As a result, it is possible to specify the current position of the endoscope distal end 3B on the bronchus image.

The virtual endoscope image generation unit 25 acquires a projection image by central projection in which a three-dimensional image on a plurality of lines of sight radially extending from a viewpoint, which is a specified position of the endoscope distal end 3B, is projected onto a predetermined projection plane. The projection image is the virtual endoscope image K0 that is virtually generated by performing imaging at the distal end position of the endoscope. As a specific central projection method, for example, a known volume rendering method can be used. It is assumed that the angle of view (range of the line of sight) of the virtual endoscope image K0 and the center (center in the projection direction) of the field of view are set in advance by user's input or the like. The generated virtual endoscope image K0 is output to the display control unit 26, and is displayed on the display 14.

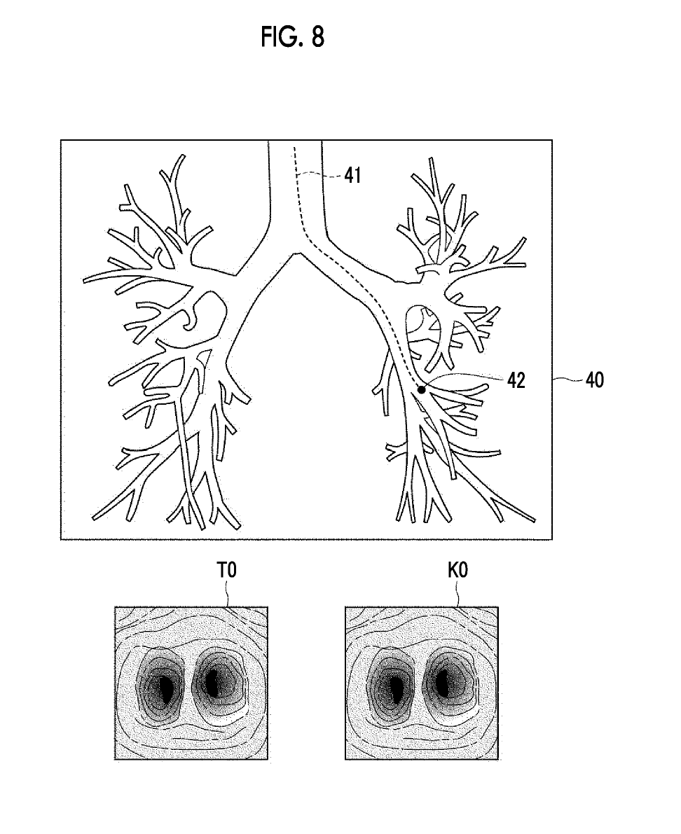

The display control unit 26 displays the real endoscope image T0 and the virtual endoscope image K0 on the display 14. FIG. 7 is a diagram showing the real endoscope image T0 and the virtual endoscope image K0 that are displayed on the display 14. As shown in FIG. 7, the real endoscope image T0 and the virtual endoscope image K0 are displayed side by side on the display 14. The display method is not limited thereto, the virtual endoscope image K0 may be displayed within the display screen of the real endoscope image T0. Alternatively, the virtual endoscope image K0 subjected to translucent processing may be superimposed on the real endoscope image T0 so as to be displayed in a blending manner. As shown in FIG. 8, a bronchus image 40 may also be displayed together with these. In this case, a path 41 of the endoscope distal end 3B and a current position 42 of the endoscope distal end 3B are displayed in the bronchus image 40.

The emphasis unit 27 emphasizes a branching structure in the real endoscope image T0 displayed on the display 14 in a case where the determination unit 24 determines that the branching structure of the bronchus is included in the real endoscope image T0.

The warning unit 28 gives a warning in a case where the determination unit 24 determines that the branching structure of the bronchus is included in the real endoscope image T0.

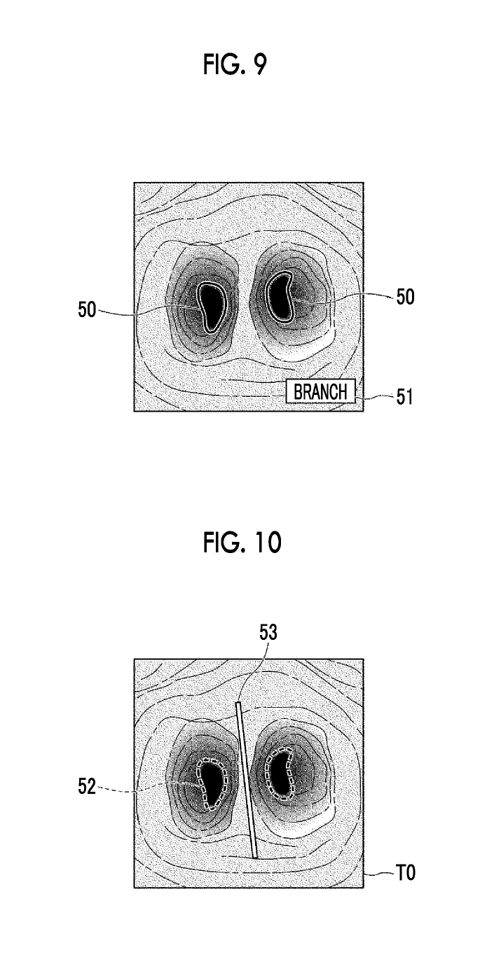

FIG. 9 is a diagram showing the real endoscope image T0 in which the branching structure is emphasized. As shown in FIG. 9, the branching structure included in the real endoscope image T0 is emphasized by giving a marker 50 that is a frame surrounding the hole. In this case, the emphasis unit 27 specifies the position of the hole in the real endoscope image T0 based on the information of the position acquired by the first evaluation value E1. In the real endoscope image T0, a warning is given by displaying the word “branch” indicating the branching structure as a warning 51.

As shown in FIG. 10, the branching structure may be emphasized by giving a dotted marker 52 around the hole of the branching structure and providing a linear marker 53 in a boundary portion. The linear marker 53 may be given only to the boundary portion. In this case, the emphasis unit 27 specifies the position of the boundary in the real endoscope image T0 based on the information of the position acquired by the second evaluation value E2. Color may be given to the marker, or the marker may be blinked. The warning may be performed not only by superposition display on the real endoscope image T0 but also by sound, or may be performed by blinking the real endoscope image T0 itself displayed on the display 14.

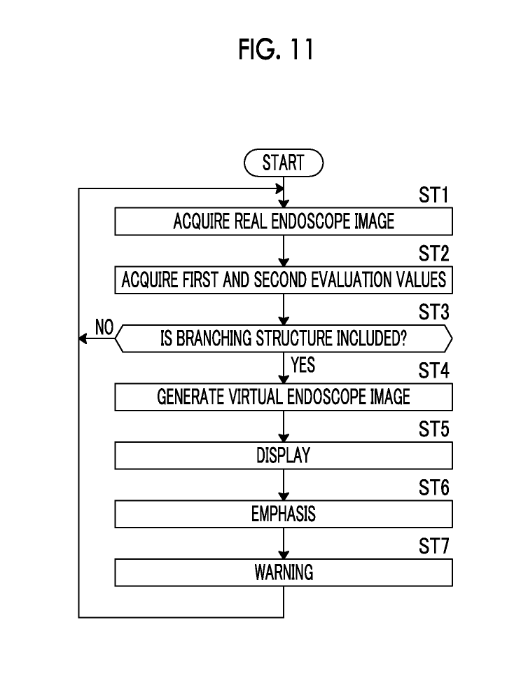

Next, a process performed in the present embodiment will be described. FIG. 11 is a flowchart showing the process performed in the present embodiment. It is assumed that the three-dimensional image V0 is acquired by the image acquisition unit 21 and is stored in the storage 13. First, the image acquisition unit 21 acquires the real endoscope image T0 (step ST1). The first evaluation value acquisition unit 22 acquires the first evaluation value E1 indicating the hole likeness of the insertion destination of the endoscope, and the second evaluation value acquisition unit 23 acquires the second evaluation value E2 indicating the boundary likeness of a plurality of bronchi at the bronchial branch (step ST2). Then, the determination unit 24 determines whether or not a branching structure is included in the real endoscope image T0 using both the first and second evaluation values E1 and E2 (step ST3). When step ST3 is negative since a branching structure is not included, the process returns to step ST1.

When step ST3 is positive since a branching structure is included, the virtual endoscope image generation unit 25 generates the virtual endoscope image K0 (step ST4). Then, the display control unit 26 displays the real endoscope image T0 and the virtual endoscope image K0 on the display 14 (step ST5). In addition, the emphasis unit 27 emphasizes the branching structure in the real endoscope image T0 (step ST6), and the warning unit 28 gives a warning regarding the branching structure (step ST7), and returns to step ST1.

Thus, in the present embodiment, the first evaluation value E1 indicating the hole likeness of the insertion destination of the endoscope and the second evaluation value E2 indicating the boundary likeness of a plurality of tubular structures at the branch of the tubular structure are acquired, and it is determined whether or not a branching structure is included in the real endoscope image T0 using both the first and second evaluation values E1 and E2. Here, the first and second evaluation values E1 and E2 remarkably indicate the feature of the branching position. Therefore, according to the present embodiment, it is possible to accurately determine whether or not a branching structure is included in the real endoscope image T0 using the first and second evaluation values E1 and E2.

In the present embodiment, the virtual endoscope image K0 showing the inner wall of the bronchus in a case where the branching structure is viewed at a position in the three-dimensional image V0 corresponding to the position of the endoscope, at which the real endoscope image T0 determined to include the branching structure is acquired, is generated from the three-dimensional image V0. Therefore, compared with a case of generating the virtual endoscope image K0 by searching for the entire three-dimensional image V0, it is sufficient to search for only the three-dimensional image V0 around the branching structure. As a result, it is possible to quickly generate the virtual endoscope image K0 with a small calculation amount.

In addition, by extracting a bronchus image from the three-dimensional image V0 and specifying the position of the endoscope distal end 3B in the extracted bronchus image, it is possible to know the current position of the endoscope distal end 3B in the bronchus. Therefore, it is possible to easily operate the endoscope.

By displaying the extracted tubular structure and specifying the position of the endoscope distal end 3B specified in the displayed tubular structure, it is possible to more easily know the current position of the endoscope distal end 3B in the tubular structure.

In addition, by emphasizing the branching structure in the case of displaying the real endoscope image T0, it becomes easy to recognize the branching structure. Therefore, it becomes easy to operate the endoscope.

In addition, by giving a warning in a case where it is determined that a branching structure is included, the user can easily recognize that the branching structure is included in the real endoscope image.

In the embodiment described above, the branching structure is emphasized in the case of displaying the real endoscope image T0. However, the real endoscope image T0 may be displayed without emphasizing the branching structure.

In the embodiment described above, a warning is given in a case where it is determined that a branching structure is included. However, no warning may be given.

In the embodiment described above, a bronchus image is extracted from the three-dimensional image V0, and the virtual endoscope image K0 is generated using the bronchus image. However, the virtual endoscope image K0 may be generated from the three-dimensional image V0 without extracting a bronchus image.

In the above embodiment, the case has been described in which the branching structure determination apparatus of the present invention is applied to the observation of the bronchus. However, without being limited thereto, the present invention can also be applied to a case of observing a tubular structure having a branching structure, such as a blood vessel, with an endoscope.

Hereinafter, the operational effect of the embodiment of the present invention will be described.

By generating, from the three-dimensional image, a virtual endoscope image showing the inner wall of the tubular structure in a case where the branching structure is viewed at a position in the three-dimensional image corresponding to the position of the endoscope where the real endoscope image determined to include the branching structure is acquired, it is sufficient to search for only the three-dimensional image around the branching structure compared with the case of generating the virtual endoscope image by searching for the entire three-dimensional image. As a result, it is possible to quickly generate the virtual endoscope image with a small calculation amount.

By extracting the tubular structure from the three-dimensional image and specifying the position of the endoscope in the extracted tubular structure, it is possible to know the current position of the endoscope in the tubular structure. Accordingly, it is possible to easily operate the endoscope.

By displaying the extracted tubular structure and specifying the position of the endoscope specified in the displayed tubular structure, it is possible to more easily know the current position of the endoscope in the tubular structure.

What is claimed is:

1. A branching structure determination apparatus, comprising:
real endoscope image acquisition unit for acquiring a real endoscope image that is generated by performing imaging using an endoscope inserted into a tubular structure having a branching structure in a subject and that shows an inner wall of the tubular structure;
first evaluation value acquisition unit for acquiring a first evaluation value, which indicates hole likeness of an insertion destination of the endoscope in the tubular structure, from the real endoscope image;
second evaluation value acquisition unit for acquiring a second evaluation value, which indicates boundary likeness of a plurality of tubular structures at a branch of the tubular structure, from the real endoscope image; and
determination unit for determining whether or not the branching structure is included in the real endoscope image using both the first and second evaluation values.

2. The branching structure determination apparatus according to claim 1,
wherein the determination unit acquires a candidate for a hole into which the endoscope is to be inserted based on the first evaluation value, acquires a candidate for a boundary of a plurality of tubular structures at a branch of the tubular structure based on the second evaluation value, and determines whether or not the branching structure is included in the real endoscope image based on the hole candidate and the boundary candidate.

3. The branching structure determination apparatus according to claim 2,
wherein the determination unit calculates a weighted average of the first evaluation value, which is the hole candidate, and the second evaluation value, which is the boundary candidate, and determines that the branching structure is included in the real endoscope image in a case where the weighted average is equal to or greater than a predetermined threshold value.

4. The branching structure determination apparatus according to claim 1,
wherein the first evaluation value acquisition unit calculates the first evaluation value using a learning result acquired by machine-learning the hole likeness of the insertion destination of the endoscope.

5. The branching structure determination apparatus according to claim 1,
wherein the second evaluation value acquisition unit calculates the second evaluation value using a learning result acquired by machine-learning the boundary likeness of the plurality of tubular structures.

6. The branching structure determination apparatus according to claim 1, further comprising:
real endoscope image display unit for displaying the real endoscope image.

7. The branching structure determination apparatus according to claim 6, further comprising:
emphasis unit for emphasizing the branching structure in the displayed real endoscope image in a case where it is determined that the branching structure is included.

8. The branching structure determination apparatus according to claim 7,
wherein the emphasis unit emphasizes the branching structure by giving a marker to at least one of a hole of the insertion destination of the endoscope in the tubular structure or a boundary of the plurality of tubular structures.

9. The branching structure determination apparatus according to claim 1, further comprising:
warning unit for giving a warning in a case where it is determined that the branching structure is included.

10. The branching structure determination apparatus according to claim 1, further comprising:
three-dimensional image acquisition unit for acquiring a three-dimensional image including the tubular structure of the subject; and
virtual endoscope image generation unit for generating a virtual endoscope image, which shows an inner wall of the tubular structure in a case where the branching structure is viewed at a position in the three-dimensional image corresponding to a position of a distal end of the endoscope where a real endoscope image determined to include the branching structure is acquired, from the three-dimensional image in a case where it is determined that the branching structure is included.

11. The branching structure determination apparatus according to claim 10, further comprising:
virtual endoscope image display unit for displaying the virtual endoscope image.

12. The branching structure determination apparatus according to claim 10,
wherein the virtual endoscope image generation unit extracts the tubular structure from the three-dimensional image, and specifies a position of a distal end of the endoscope in the extracted tubular structure.

13. The branching structure determination apparatus according to claim 12, further comprising:
tubular structure display unit for displaying an image of the extracted tubular structure.

14. The branching structure determination apparatus according to claim 13,
wherein the tubular structure display unit further displays the specified position of the distal end of the endoscope in the displayed tubular structure.

15. A branching structure determination method, comprising:
acquiring a real endoscope image that is generated by performing imaging using an endoscope inserted into a tubular structure having a branching structure in a subject and that shows an inner wall of the tubular structure;
acquiring a first evaluation value, which indicates hole likeness of an insertion destination of the endoscope in the tubular structure, from the real endoscope image;
acquiring a second evaluation value, which indicates boundary likeness of a plurality of tubular structures at a branch of the tubular structure, from the real endoscope image; and
determining whether or not the branching structure is included in the real endoscope image using both the first and second evaluation values.

16. A non-transitory computer-readable recording medium having stored therein a branching structure determination program causing a computer to execute:
a step of acquiring a real endoscope image that is generated by performing imaging using an endoscope inserted into a tubular structure having a branching structure in a subject and that shows an inner wall of the tubular structure;
a step of acquiring a first evaluation value, which indicates hole likeness of an insertion destination of the endoscope in the tubular structure, from the real endoscope image;
a step of acquiring a second evaluation value, which indicates boundary likeness of a plurality of tubular structures at a branch of the tubular structure, from the real endoscope image; and
a step of determining whether or not the branching structure is included in the real endoscope image using both the first and second evaluation values.
